(19) United States
(12) Patent Application Publication
(10) Pub. No.: US 2024/0270672 A1
(43) Pub. Date: Aug. 15, 2024

(54) Process For The Manufacture Of A Potassium Salt Of A Benzoquinone

(71) Applicant: CMBlu Energy AG, Alzenau (DE)

(72) Inventors: Peter GEIGLE, Alzenau (DE); Doris NEUMANN, Offenbach (DE); Christian SCHNEIDER, Aschaffenburg (DE); Markus Richard HARTMANN, Seligenstadt (DE); Silke SCHLEICH, Alzenau (DE); Cesare SAVARINO, Budingen (DE)

(21) Appl. No.: 18/289,595

(22) PCT Filed: May 6, 2021
(86) PCT No.: PCT/EP2021/062040

§ 371 Date: Nov. 6, 2023

(51) Int. Cl. C07C 46/06

(52) U.S. Cl. CPC C07C 46/06

(57) ABSTRACT

The present invention relates to the manufacture of benzoquinones, in particular to the manufacture of a potassium salt of a benzoquinone, such as 2,5-dihydroxy-1,4-benzoquinone di-potassium salt. The potassium salt of a benzoquinone is obtained by oxidation of a hydroquinone using potassium hydroxide and hydrogen peroxide, which results in an efficient process with high yields.

The present invention relates to the field of the manufacture of benzoquinones, in particular to the manufacture of a potassium salt of a benzoquinone, such as 2,5-dihydroxy-1,4-benzoquinone di-potassium salt. In particular, the potassium salt of a benzoquinone is obtained by oxidation of a hydroquinone using potassium hydroxide and hydrogen peroxide.

Benzoquinones, such as 2,5-dihydroxy-1,4-benzoquinone, and their salts are important intermediates in chemical synthesis, for example in the synthesis of dihydroxyphenazines which are promising candidates as electroactive materials in electrical energy storage. Accordingly, various processes for the manufacture of benzoquinones or salts thereof are known in the art.

For example, R. G. Jones et al. (R. G. Jones, H. A. Shonle, J. Am. Chem. Soc. 1945, 67, 1034-1035.) describe the preparation of 2,5-dihydroxy-1,4-benzoquinone by oxidation of hydroquinone in sodium hydroxide solution with a hydrogen peroxide solution of 27 wt %. This process is designed to yield <80% of the protonated 2,5-dihydroxy-1,4-benzoquinone. The reaction conditions of Jones et al. involve considerable safety risks.

Viault et al. (G. Viault et al., Eur. J. Org. Chem. 2011, 7, 1233-1241.) disclose a process relying on a reaction mixture with an extensive excess of 6.3 equivalents of hydrogen peroxide and 9.6 equivalents of sodium hydroxide relative to hydroquinone which are heated to 45° C. for 2 hours. This process results in relatively unstable 2,5-dihydroxy-1,4-benzoquinone in moderate yields of 70% and requires extensive cooling with ice.

Sudhakar et al. (Ch. Sudhakar et al., Der Chemica Sinica 2016, 7(2), 82-85.) oxidize hydroquinone in an aqueous solution of sodium hydroxide and hydrogen peroxide at 45-50° C. for 2 h and designed a work-up that results in 2,5-dihydroxy-1,4-benzoquinone.

While most oxidation processes of hydroquinone to a benzoquinone (salt) rely on aqueous solutions of sodium hydroxide and hydrogen peroxide, as described above, Prakash et al. (D. Prakash et al., Acta Ciencia Indica, Chemistry 2003, 29(2), 113-116.) describe a synthetic procedure with a solvent mixture based on water and ethanol without any oxidant and excessive heating to reflux for 30 minutes. However, the organic (co-)solvents used in this process lead to ecologic and commercial challenges and it remains unclear which oxidant drives the chemical transformation.

In summary, all of the above described processes result in yields below 80% and use partly organic solvents or rely on a work-up that yields 2,5-dihydroxy-1,4-benzoquinone, which is less stable than, for example, a potassium salt thereof, such as 2,5-dihydroxy-1,4-benzoquinone di-potassium salt (DKBQ).

Therefore, there is an urgent need of production processes, which are commercially more attractive, result in higher yields or avoid the relatively unstable 2,5-dihydroxy-1,4-benzoquinone.

In view of the above, it is the object of the present invention to provide a novel production process for a potassium salt of a benzoquinone, which overcomes the above-mentioned drawbacks.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%, preferably x±5%, more preferably x±2%, even more preferably x±1%.

As used herein, the term “hydroxyl” refers to a —OH group, preferably including all of its protonation states, such as —O⁻.

Identical labels for symbols or groups used in distinct formulae, usually refer to the same definition of said group or symbol. In other words, the definition of said group or symbol provided in the context of one specific formula typically also applies to other formulae using the same label.

The expression “essentially no . . . is present”, as used herein, means that the respective substance/compound may ideally not be present. Nevertheless, minor traces may be present, for example in an amount not exceeding 5% by weight, preferably not exceeding 3% by weight, more preferably not exceeding 2% by weight, even more preferably not exceeding 1% by weight, still more preferably not exceeding 0.5% by weight.

As used herein, the term “equivalents” usually refers to molar equivalents. A molar equivalent is the ratio of the moles of one compound to the moles of another (reference compound, for example the compound of formula (II)). In other words, an equivalent is the number by which the arbitrary amount (moles) of a reference compound (e.g. the compound of formula (II)) needs to be multiplied, to calculate the amount of a substance used as reactant in a chemical reaction. For example, the reaction of 10 “equivalents” of KOH or H₂O₂ with the compound of the formula (II) in the oxidation reaction of the process of the present invention refers to the reaction of 10 moles of KOH or H₂O₂ with 1 mol of the compound of the formula (II).

As used herein, the expression “at the beginning of the oxidation” refers to the time point, when the reaction mixture is just completed (including the compound of formula (II), potassium hydroxide (KOH) and hydrogen peroxide (H₂O₂)); and the reaction therefore begins.

### Process for the Manufacture of a Potassium Salt of a Benzoquinone

In a first aspect the present invention provides a process for the manufacture of a compound according to formula (I)

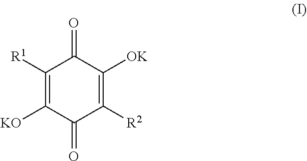

by alkaline oxidation of a compound according to formula (II)

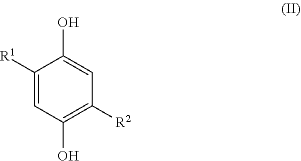

using potassium hydroxide (KOH) and hydrogen peroxide (H₂O₂),
wherein R¹ and R² are independently selected from the group consisting of —OR^{x}, —NO₂, —(NR^{x})R^{y}, —SR^{x}, —(SO)R^{x}, —(SO₂)R^{x}, —(SO₃)R^{x}, —(CO)R^{x}, —(CO₂)R^{x}, wherein R^{x} is H or C₁₋₆ alkyl and R^{y} is H or C₁₋₆ alkyl optionally substituted with —(CO₂)R^{x}; and salts thereof.

Accordingly, the reaction equation may be expressed as follows:

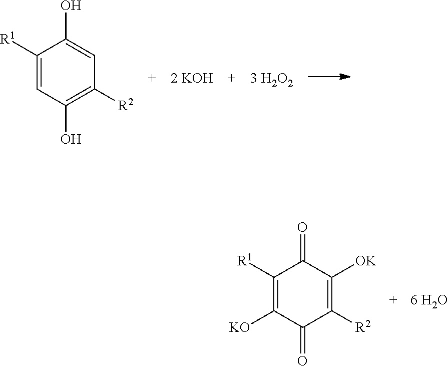

According to the present invention, the oxidation reaction of the compound of formula (II) to the compound of formula (I) occurs in the presence of potassium hydroxide (KOH) and hydrogen peroxide (H₂O₂).

The present inventors have surprisingly found that the process according to the present invention using KOH and H₂O₂ for the oxidation of the compound of formula (II) results in considerably higher yields and increased purities as compared to processes of the prior art. Moreover, the obtained product, the potassium salt of formula (II) provides increased stability and can be directly used in subsequent reactions, for example in the condensation to phenazines. In addition, the isolation of the product from the reaction mixture is particularly simple. These advantages, in particular in their combination, result in a commercially highly attractive process.

In formula (I) and (II), R¹ and R² may be the same or different. As described above, R¹ and R² are independently selected from the group consisting of —OR^{x}, —NO₂, —(NR^{x})R^{y}, —SR^{x}, —(SO)R^{x}, —(SO₂)R^{x}, —(SO₃)R^{x}, —(CO)R^{x}, —(CO₂)R^{x}, wherein R^{x} is H or C₁₋₆ alkyl and R^{y} is H or C₁₋₆ alkyl optionally substituted with —(CO₂)R^{x}; and salts thereof (including inner salts). R^{x} and R^{y} are selected independently and may be the same or different.

The term “alkyl” refers to the radical of saturated hydrocarbon groups, including linear (i.e. straight-chain) alkyl groups and branched-chain alkyl groups. The alkyl group contains from 1 to 6 carbon atoms (“C₁₋₆ alkyl”). In some embodiments, an alkyl group has 1 to 5 carbon atoms (“C₁₋₅ alkyl”). In some embodiments, an alkyl group may contain 1 to 4 carbon atoms (“C₁₋₄ alkyl”), from 1 to 3 carbon atoms (“C₁₋₃ alkyl”), or from 1 to 2 carbon atoms (“C₁₋₂ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), and hexyl (C₆) (e.g., n-hexyl).

Unless otherwise specified, each instance of an alkyl group is independently unsubstituted (an “unsubstituted alkyl”) or substituted (a “substituted alkyl”) with one or more substituents. In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. Compounds described herein contemplates any and all such combinations in order to arrive at a stable compound. Heteroatoms such as nitrogen may have hydrogen substituents and/or any suitable substituent as described herein which satisfy the valencies of the heteroatoms and results in the formation of a stable moiety. Compounds described herein are not intended to be limited in any manner by the exemplary substituents described herein.

In some embodiments, the alkyl group is an unsubstituted C₁₋₆ alkyl, e.g., —CH₃ (Me), unsubstituted ethyl (Et), unsubstituted propyl (Pr, e.g., unsubstituted n-propyl (n-Pr)), unsubstituted isopropyl (iso-Pr or i-Pr)), unsubstituted butyl (Bu, e.g., unsubstituted n-butyl (n-Bu)), unsubstituted tert-butyl (tert-Bu or t-Bu), unsubstituted sec-butyl (sec-Bu or s-Bu), or unsubstituted isobutyl (iso-Bu or i-Bu).

In certain embodiments, the alkyl group is a substituted C₁₋₆alkyl, e.g., —CF₃, Bn. Exemplary substituents may include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. Substituents may themselves be substituted. For instance, the substituents of a “substituted alkyl” may include both substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls may be further substituted with alkyls, alkenyls, alkoxys, alkylthios, aminoalkyls, carbonyl-substituted alkyls, —CF₃, —CN, and the like.

Preferably, R¹ and R² in formula (I) and (II) are independently selected from the group consisting of —H, —OH, —NH₂, —CN, —CO₂H, —SO₃H and salts thereof. More preferably, R¹ and R² in formula (I) and (II) are independently selected from the group consisting of —H, —OH and salts thereof.

As used herein, the expression “salts thereof” refers to derivatives of the disclosed compounds wherein the parent (reference) compound is modified by making salts thereof, e.g. with bases. The salts can be synthesized, e.g. from a parent compound which contains an acidic moiety, by conventional chemical methods. Generally, such salts can be prepared by reacting the free acid forms of these compounds with a sufficient amount of the appropriate base, for example in water or in an organic diluent like ether, ethyl acetate, ethanol, isopropanol, or acetonitrile, or a mixture thereof. Alternatively, salts can be prepared by ion exchange, for example by treating aqueous solutions of the parent compounds (free acid or salt form) with a cation exchanger.

In some embodiments, the salt thereof may be an inner salt. As used herein, the expression “inner salt” refers to derivatives of the disclosed compounds wherein the parent (reference) compound contains an acidic and a basic moiety which react with each other forming a cationic and an anionic structure in one molecule. It is to be understood that the term “inner salt” refers to a zwitterionic structure within one molecule. For example, an amino acid may be in the form of an inner salt (zwitterion).

Preferred salts are alkali-salts. As used herein, the term “alkali salt” refers to any salt of an alkali metal. Alkali metals include lithium, sodium, potassium, rubidium, cesium and francium. Alkali salts usually exhibit a polar character and excellent solubility in water and aqueous solutions. Accordingly, R¹ and R² may be independently selected from the group consisting of —OR^{x}, —NO₂, —(NR^{x})R^{y}, —SR^{x}, —(SO)R^{x}, —(SO₂)R^{x}, —(SO₃)R^{x}, —(CO)R^{x}, —(CO₂)R^{x}, wherein R^{x} and R^{y} are selected independently and R^{x} is H or C₁₋₆ alkyl and R^{y} is H or C₁₋₆ alkyl optionally substituted with —(CO₂)R^{x}; and salts thereof, including inner salts. Preferably, R¹ and R² in formula (I) and (II) are independently selected from the group consisting of —H, —OH, —NH₂, —CN, —CO₂H, —SO₃H and alkali salts thereof. More preferably, R¹ and R² in formula (I) and (II) are independently selected from the group consisting of —H, —OH and alkali salts thereof.

Most preferably, each of R¹ and R² is —H. Accordingly, the compound of formula (II) may be hydroquinone, as shown in formula (IIa):

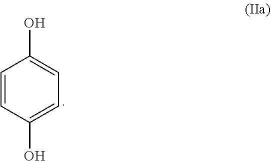

Accordingly, the compound of formula (I) may be 2,5-dihydroxy-1,4-benzoquinone di-potassium salt (DKBQ), as shown in formula (Ia):

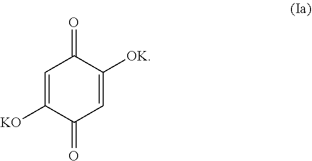

Accordingly, the reaction equation is preferably as follows:

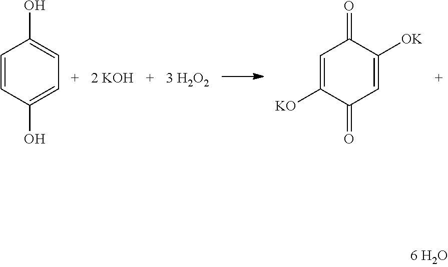

In some embodiments, the process comprises the following steps:
(1) providing an aqueous solution of KOH;
(2) adding the compound of formula (II) to the aqueous KOH solution provided in step (1);
(3) adding H₂O₂ to the reaction mixture obtained in step (2); and
(4) maintaining the reaction mixture at a temperature of at least 45° C.

KOH is preferably added in excess. The amount of KOH may depend on the amount of the compound of formula (II), in particular hydroquinone. Preferably, (at the beginning of the oxidation) the amount of KOH included in the reaction process is at least 8 equivalents relative to the amount of the compound of formula (II), in particular hydroquinone. More preferably, (at the beginning of the oxidation) the amount of KOH included in the reaction process is at least 9 equivalents relative to the amount of the compound of formula (II), in particular hydroquinone. Even more preferably, (at the beginning of the oxidation) the amount of KOH included in the reaction process is at least 9.8 equivalents relative to the amount of the compound of formula (II), in particular hydroquinone; for example (at the beginning of the oxidation) the amount of KOH included in the reaction process may be about 9.8-12.2 equivalents relative to the amount of the compound of formula (II), in particular hydroquinone. Still more preferably, (at the beginning of the oxidation) the amount of KOH included in the reaction process is (at least) about 10 equivalents relative to the amount of the compound of formula (II), in particular hydroquinone.

Without being bound to any theory, the inventors have found that such high amounts of KOH result in particularly high yields of the reaction product (the compound of formula (I)). While conventionally sodium hydroxide (NaOH) is typically used as base, the present inventors have surprisingly found that the yields obtained with KOH (instead of NaOH) as base are considerably higher than the yields, which can be obtained with NaOH in a process, which is otherwise essentially the same.

In the process of the invention, the potassium hydroxide (KOH) is preferably provided as aqueous solution, for example as KOH dissolved in water. In some embodiments, in particular in a batch process, an aqueous KOH solution of at least 40 wt % KOH, preferably at least 43 wt % KOH, more preferably at least 45 wt % KOH, even more preferably at least 47 wt % KOH may be provided, still more preferably at least 50 wt % KOH may be provided, e.g. in step (1) of the (batch) process as described herein.

In some embodiments, in particular in a continuous process, an aqueous KOH solution of at least 40 wt % KOH, preferably 45±5 wt % KOH, more preferably 45±4 wt % KOH, even more preferably 45±3 wt % KOH, still more preferably 45±2 wt % KOH, most preferably 45±1 wt % KOH may be provided, e.g. in step (1) of the (continuous) process as described herein.

Optionally, further KOH may be added before, during or after the addition of the compound of formula (II) (step (2) of the process as described above); for example as solid, e.g. having a purity of at least 80% (e.g. about 85%). Moreover, further KOH may be added before, during or after the addition of H₂O₂ (step (3) of the process as described above), for example as solid, e.g. as described above. In some embodiments, KOH may be continuously added during the process, in particular if the process is performed as continuous process. For example, KOH may be added in parallel to the addition of H₂O₂, e.g. in a step-wise manner.

Preferably, the concentration of KOH at the beginning of the oxidation is 35-61 wt %, more preferably 37-57 wt %, even more preferably 40-55 wt % in the reaction mixture. Most preferably, the concentration of KOH at the beginning of the oxidation is about 45 wt % (±1 wt %; e.g., for a continuous process) or at least 50 wt % (e.g., for a batch process).

In some embodiments, KOH is dosed continuously throughout the reaction to maintain a minimum potassium hydroxide concentration of about 45 wt % (e.g. 1 wt %) throughout the reaction. To this end, a flow system employing two or more parallel cartridges of potassium hydroxide may be used.

The amount of H₂O₂ included in the reaction process is preferably at least 2.5 equivalents relative to the amount of the compound of formula (II), in particular hydroquinone. More preferably, the amount of H₂O₂ is at least 2.7 equivalents relative to the amount of the compound of formula (II), in particular hydroquinone, even more preferably at least 3.0 equivalents relative to the amount of the compound of formula (II), in particular hydroquinone. In some embodiments, the amount of H₂O₂ included in the reaction process is 2.7 to 3.52 equivalents relative to the amount of the compound of formula (II), in particular hydroquinone; preferably 2.9-3.4 equivalents relative to the amount of the compound of formula (II), in particular hydroquinone; more preferably 3.0-3.3 equivalents (e.g. about 3.1 or 3.25 equivalents) relative to the amount of the compound of formula (II), in particular hydroquinone.

The hydrogen peroxide (H₂O₂) is preferably provided as aqueous solution, for example as H₂O₂ dissolved in water. In some embodiments, the H₂O₂ is provided in an aqueous solution of at least 27 wt % H₂O₂, preferably 30 wt % H₂O₂, more preferably at least 35 wt % H₂O₂; e.g. in step (3) of the process as described above. Even more preferably, the H₂O₂ is provided in an aqueous solution of at least 40 wt % or at least 45 wt % H₂O₂, e.g. in step (3) of the process as described above. Still more preferably, the H₂O₂ is provided in an aqueous solution of (at least) about 50 wt % H₂O₂, e.g. in step (3) of the process as described above.

Preferably, the H₂O₂ (aqueous solution) is added at a low rate, e.g. in small portions (for example dropwise). The addition rate can vary and depends amongst other parameters particularly on the size of the reaction vessel and stirring rate. Preferably the H₂O₂ (aqueous solution) is added (under stirring) at elevated temperatures, i.e. at temperatures above 25° C., preferably at a temperature of 45-60° C., more preferably 47-57° C., even more preferably 49-56° C., still more preferably 50-55° C.

In some embodiments, the hydrogen peroxide (H₂O₂) may be added via a venturi nozzle. The use of a venturi nozzle was found to advantageously decrease side-product formation in the overall reaction.

With the addition of hydrogen peroxide, the reaction, as described above, starts and the formation of the product (compound of formula (I), in particular 2,5-dihydroxy-1,4-benzoquinone di-potassium salt (DKBQ)), begins. The progress of the reaction may be monitored analytically. For example, small samples of the reaction mixture may be analyzed, e.g., by HPLC.

The reaction temperature (at which the above-mentioned oxidation occurs) is preferably at least 45° C., more preferably at least 47° C., even more preferably at least 50° C. Preferably, the reaction temperature does not exceed 60° C. A reaction temperature above 60° C. may result in a larger relative amount of undesired by-products. In some embodiments, the reaction temperature is 45-60° C., preferably 47-57° C., more preferably 49-56° C., even more preferably 50-55° C. For example, the reaction temperature may be 52-55° C., such as about 53° C. As the reaction is exothermic, said temperature may be obtained by cooling. To maintain the temperature as described above, a tube bundle heat exchanger may be used. Thereby, the overall reaction time may be reduced.

The reaction mixture may be stirred in any one of steps (1)-(4). In particular, the mixture may be stirred in steps (2) and (4). In some embodiments, the mixture may be stirred throughout the entire reaction (steps (1)-(4)).

In some embodiments, the sum of the weight of:
(i) the compound of formula (II);
(ii) KOH;
(iii) H₂O₂; and
(iv) water
   is more than 95%, preferably more than 98%, more preferably more than 99% of the total weight of the reaction mixture at the beginning of the oxidation.

In particular, the reaction mixture (at the beginning of the oxidation) may consist essentially of:
the compound of formula (II), in particular hydroquinone;
(an aqueous solution of) KOH; and
(an aqueous solution of) H₂O₂.

In this context, water may be provided by the aqueous solution(s). Optionally, (further) water may be added to the compound of formula (II), KOH and H₂O₂. It is understood that during the (ongoing) reaction (oxidation) increasing amounts of the reaction product will be present.

Additional compounds may not be required for the reaction as described herein. Accordingly, for example essentially no sodium hydroxide (NaOH) may be present during the oxidation. Moreover, essentially no organic solvent may be present during the oxidation. In some embodiments, essentially no (organic) acid may be present during the oxidation. In some embodiments, essentially no organic solvent, such as ethanol, may be present during the oxidation.

The reaction may be performed in a jacketed reactor. The process of the invention may be performed as batch process or as continuous process.

In some embodiments the process is a batch process.

In step (1) of an exemplified batch process, an aqueous solution of KOH as described above may be provided, e.g. by dissolving KOH in water. Additional KOH may optionally be added and dissolved in the aqueous KOH solution. For example, at first a 50 wt % aqueous KOH solution may be provided to which further KOH (e.g. having a purity of at least 80%, such as 85%) is added to obtain an aqueous solution of about 55 wt %. The amount of KOH may depend on the amount of the compound of formula (II), in particular hydroquinone. For example, at least 8 equivalents KOH (relative to the compound of formula (II), in particular hydroquinone), such as about 10 equivalents KOH may be used.

In step (2) of the exemplified batch process, the compound of formula (II), in particular hydroquinone, is added to the aqueous KOH solution. The compound of formula (II), in particular hydroquinone, may be added in portions to prevent the neutralization temperature to heat the solution above 50° C.

In step (3) of the exemplified batch process, H₂O₂ is added, preferably as aqueous solution as described above, for example as about 50 wt % aqueous solution. The amount of H₂O₂ is preferably as described above, for example about 3.25 equivalents relative to the compound of formula (II), in particular hydroquinone. The aqueous hydrogen peroxide solution may be added in a constant flow. Thereby, the temperature of the reaction mixture may be maintained as described above, e.g. at 50-55° C., for example by cooling.

In step (4), the temperature may be maintained as described above, in particular at at least 45° C., preferably 47-57° C., more preferably 49-56° C., even more preferably 50-55° C. The reaction may be performed until essentially no compound of formula (II), in particular hydroquinone, is detectable in the reaction mixture. As described above, the progress of the reaction may be monitored analytically. For example, small samples of the reaction mixture may be analyzed, e.g., by HPLC. In some embodiments, the reaction temperature as described above may be maintained for at least 10, 15, 20, 25, or 30 min after the addition of H₂O₂ is finalized. In some embodiments, the temperature is maintained in step (4) as described above for at least 30 min. Preferably, the temperature is maintained in step (4) as described above for at least 35 or 40 min. After step (4), the mixture may be allowed to cool, e.g. to ambient temperature, for example to about 25-30° C. Thereafter, the product may be optionally washed and isolated.

Throughout the reaction (steps (1)-(4)), the mixture may be stirred.

In some embodiments, the process is a continuous process.

In step (1) of an exemplified continuous process, an aqueous solution of KOH as described above may be provided, e.g. by dissolving KOH in water. For example, at first a 45±1 wt % aqueous KOH solution may be provided. The amount of KOH may depend on the amount of the compound of formula (II), in particular hydroquinone. For example, at least 8 equivalents KOH (relative to the compound of formula (II), in particular hydroquinone), such as about 10 equivalents KOH may be used.

In step (2) of the exemplified continuous process, the compound of formula (II), in particular hydroquinone, is added to the aqueous KOH solution. The compound of formula (II), in particular hydroquinone, may be added in a single portion. Thereafter, further KOH may be added.

In step (3) of the exemplified continuous process, H₂O₂ is added, preferably as aqueous solution as described above, for example as about 50 wt % aqueous solution. The amount of H₂O₂ is preferably as described above, for example about 3.1 equivalents relative to the compound of formula (II), in particular hydroquinone. The aqueous hydrogen peroxide solution may be added, for example, at a rate of 3.9 mL/min. Preferably, a venturi nozzle is used to add the H₂O₂. The temperature of the reaction mixture may be maintained as described above, e.g. at 50-55° C., in particular at about 53° C. In parallel to the addition of H₂O₂, further KOH may be added, for example in a step-wise manner (in several small portions).

In general, KOH may be added continuously during the reaction. Thereby, a minimum potassium hydroxide concentration of at least 40 wt % may be maintained throughout the reaction. For the addition of KOH, in particular in a continuous process, a flow system may be used, which may employ one or more (e.g., two) cartridges of potassium hydroxide, wherein more than one cartridge may be arranged/used in parallel.

In step (4), i.e. after finalization of the hydrogen peroxide addition, the reaction may be kept at a temperature as described above, in particular at at least 45° C., preferably 47-57° C., more preferably 49-56° C., even more preferably 50-55° C., such as at about 53° C. To control the temperature, a tube bundle heat exchanger may be used. As described above, the reaction may be performed until essentially no compound of formula (II), in particular hydroquinone, is detectable in the reaction mixture. As described above, the progress of the reaction may be monitored analytically. In some embodiments, the reaction temperature as described above may be maintained for at least 10, 15, 20, 25, or 30 min after the addition of H₂O₂ is finalized.

In some embodiments, the temperature is maintained in step (4) as described above for at least 30 min. Preferably, the temperature is maintained in step (4) as described above for at least 45 min. More preferably, the temperature is maintained in step (4) as described above for at least 1 hour after addition of H₂O₂ is finalized. Thereafter, the temperature control may be turned off. Optionally, the reaction mixture may be stirred for further 1, 2, 3, 4, 5, 6, 7, 8, 9, 10, 11, 12, 13, 14, 15, 16, 17, 18, 19, 20 or more hours (e.g., about 17 hours). Thereafter, the product may be isolated, e.g. without any further washing step.

The process of the invention may further comprise a step of isolation of the product from the reaction medium (mother liquor), in particular. As described above, the reaction may be performed until essentially no compound of formula (II), in particular hydroquinone, is detectable in the reaction mixture. As described above, the progress of the reaction may be monitored analytically. Usually, the product may be isolated at the end of or after the oxidation reaction, i.e. when essentially no compound of formula (II), in particular hydroquinone, is detectable in the reaction mixture. In some embodiments the reaction mixture may be allowed to cool (or it may be actively cooled), e.g. to a temperature of 25-30° C. or less, before the product is isolated.

For isolation of the product (the compound of formula (I)), in particular DKBQ, the reaction mixture is preferably filtered, more preferably vacuum filtration is used. The resulting filter cake may be directly used (e.g., for the synthesis of phenazines or other chemical syntheses), i.e. without additional purification steps. In some embodiments, e.g. in a batch process, the filter cake may be subdued to a washing step (using, for example, KOH solution, such as 50 wt % KOH solution).

In the continuous process, the reaction product (compound of formula (I), e.g. DKBQ) may be continuously removed. The remaining reaction medium (mother liquor), which is obtained when the reaction product is removed/isolated, may be supplemented, for example, with KOH (e.g. using one or more (e.g., two) cartridges of potassium hydroxide) and/or compound of formula (II) (hydroquinone). Thereafter, the (supplemented) mother liquor may be used (again) in the reaction (oxidation) process. To this end, the (supplemented) mother liquor may be fed into the reactor again. In addition, H₂O₂ may be added in the reactor.

In general, after isolation of the product, the remaining reaction medium (mother liquor) may be recycled, i.e. recovered and re-used, in particular in the (batch or continuous) process of the present invention as described above. Thereby, the non-reacted reagents in the mother liquor, in particular KOH, can be recovered. Recycling/re-use of the reaction medium (mother liquor) is particularly eco-friendly and cost-efficient.

In some embodiments, the recovered reaction medium (mother liquor) may be directly re-used in a (further) process according to the present invention. In other embodiments, the recovered reaction medium (mother liquor) may undergo a treatment step before it is re-used in a (further) process according to the present invention.

For example, the recovered reaction medium (mother liquor) may be treated (mixed) with activated carbon (which is removed before further use of the mother liquor, e.g. by filtration).

In some embodiments, the mother liquor is mixed with at least 1 wt % activated carbon, preferably at least 2 wt % activated carbon, more preferably at least 3 wt % activated carbon, even more preferably at least 4 wt % activated carbon, particularly preferably about 5 wt % activated carbon. The mother liquor may be mixed with the activated carbon for at least 30 min, preferably at least 45 min, more preferably at least 1 h; for example at a temperature of at least 15° C., preferably at least 20° C., more preferably at least 30° C., such as about 50° C.

In general, the mother liquor contains KOH, but usually at lower concentrations as compared to the (preferred) equivalents/concentrations of KOH as described herein above. Furthermore, usually essentially no compound of formula (II), in particular hydroquinone, may be present anymore. Accordingly, (at least) the compound of formula (II), in particular hydroquinone, needs to be added to the mother liquor for re-use. Furthermore, KOH and/or H₂O₂ may be added to the mother liquor to obtain the (preferred) equivalents/concentrations of KOH and/or H₂O₂ as described herein above. Thereby, the amount of KOH to be added is usually considerably lower as compared to the pristine chemicals used in the “first” cycle of the reaction process.

In some embodiments, after isolation of the product, the water in the reaction medium (mother liquor) may be decreased/removed (e.g., evaporated) and, thereafter, the resulting reaction medium may be used in a (further) process according to the invention. To this end, for example distillation may be performed, e.g. under reduced pressure. Optionally, the mother liquor may be filtered after decreasing the water in the mother liquor. With the decrease/removal of water the KOH and/or H₂O₂ concentration may be increased and, thus, adjusted to the (preferred) equivalents/concentrations of KOH and/or H₂O₂ as described herein above, such that no further addition of KOH and/or H₂O₂ may be required. However, in some embodiments, KOH and/or H₂O₂ may be added to the mother liquor also after the water in the reaction medium (mother liquor) is decreased/removed.

The (treated) mother liquor, e.g. with the adjusted KOH concentration as described herein, may be provided in step (1) of the process as described above, such that the remaining steps (2)-(4) may be performed as described above. In some embodiments, less H₂O₂ may be added in step (3) to obtain the desired concentrations as described above. In other embodiments, essentially the same amount of H₂O₂ may be added in step (3) as described above.

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Oxidation of hydroquinone to 2,5-dihydroxy-1,4-benzoquinone di-potassium salt (DKBQ)

For the manufacture of 2,5-dihydroxy-1,4-benzoquinone di-potassium salt (DKBQ), hydroquinone was oxidized using KOH and H₂O₂ according to the following reaction scheme:

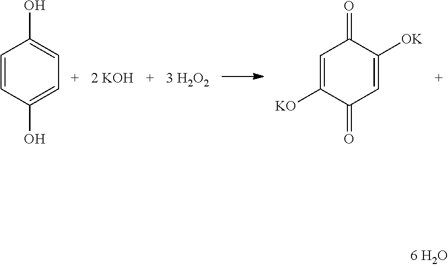

To this end, hydroquinone was mixed with a 50-55 wt % aqueous solution of potassium hydroxide (KOH). When the hydroquinone is dissolved, 50 wt % solution of hydrogen peroxide is added. The ratio of KOH and hydroquinone was about 10 equivalents (eq) KOH relative to hydroquinone (for the total KOH included in the reaction). The reaction mixture is stirred, e.g. for at least 30 min after addition of H₂O₂, preferably at a temperature of about 50-55° C. To obtain 2,5-dihydroxy-1,4-benzoquinone di-potassium salt (DKBQ) from the resulting suspension, the suspension is filtered, e.g. by vacuum filtration. Thereby, DKBQ was obtained in a yield of about 95%.

The process may be performed as batch process or as continuous (conti) process. In the following an exemplified batch process as well as an exemplified continuous process will be described:

### Batch Process

In a jacketed reactor with stirrer 4488.8 g potassium hydroxide (50 wt % aqueous solution) and additional 660.1 g of potassium hydroxide (purity 85 wt %) were provided and cooled to 30° C. 550.6 g of hydroquinone were added in a stepwise manner keeping the reactor temperature below 50° C. In a constant flow, 1105.3 g of an aqueous, 50 wt % solution of hydrogen peroxide was added under excessive cooling of the reaction mixture to keep the temperature between 50-55° C. After completed addition of the hydrogen peroxide solution the reaction mixture was stirred at 52-55° C. for further 40 minutes and subsequently cooled to 25-30° C. The corresponding suspension was filtered by vacuum filtration and sucked dry to remove residues of the mother liqueur. The resulting filter cake was optionally washed with an aqueous 50 wt % potassium hydroxide solution. Orange-red water-moist needle-shaped crystals of 2,5-dihydroxy-1,4-benzoquinone dipotassium salt were obtained in yield of >95%. An inherently high crystallinity of the product and surprisingly high yields were observed.

### Conti-Ready Process:

2.99 L potassium hydroxide solution (50 wt %) was charged into a 10 L double jacket reactor and 551 g hydroquinone were added in one portion resulting a temperature rise to about 35° C. The stirrer was turned on and further 61.7 g potassium hydroxide were added. For heating the mantle temperature was set to 55° C. and reduced when the mixture reaches 51-52° C. 929 mL 50 wt % hydrogen peroxide solution were added at a rate of 3.9 mL/min, while the reaction mixture was kept at 53° C. At about the same time, 1338 g potassium hydroxide were added step-wise in 24 portions over the addition time of the hydrogen peroxide. After finalization of the hydrogen peroxide addition, the reaction was kept at about 53° C. for one additional hour before the temperature control was turned off and the reaction was optionally stirred for about further 17 hours. The product suspension was then filtered by vacuum filtration. The obtained red crystals (DKBQ) can be directly used in further reactions, e.g. condensations to phenazines, without any additional purification steps.

### Example 2: Influence of the Amount of KOH

To investigate the influence of the amount of KOH on the resulting yield of DKBQ, the batch process as described in Example 1 was modified regarding the total KOH equivalents relative to hydroquinone by step-wise variation of the amount of KOH relative to hydroquinone from 4 to 10 equivalents of KOH. The total volume of solvent was kept constant. Results are shown in Table 1 below:

TABLE 1
Influence of the employed amount of KOH on the isolated
yield of the oxidation of hydroquinone to 2,5-dihydroxy-
1,4-benzoquinone di-potassium salt.

| Eq. KOH relative | |
|---|---|
| to hydroquinone | Isolated Yield [%] |
| 10 | 95 |
| 9 | 85 |
| 8 | 70 |
| 6 | 31 |
| 4 | 0 |

The results show that the yield increased with increasing KOH equivalents (relative to hydroquinone) with 8 eq KOH resulting in a yield of 70% and higher amounts of KOH even further increasing the yield of DKBQ.

### Example 3: Influence of NaOH vs. KOH

As the conventional oxidation of hydroquinone for 2,5-dihydroxy-1,4-benzoquinone production relies on NaOH, the effect on NaOH vs. KOH was investigated. To this end, the batch process as described in Example 1 was modified regarding the type of base that was employed during the reaction protocol (KOH vs. NaOH). In addition, the equivalents of NaOH (relative to hydroquinone) were varied, similarly as described in Example 2 above for KOH. The total volume of solvent was kept constant. Results are shown in Table 2 below:

TABLE 2
Influence of KOH vs. NaOH on the isolated
yield of the oxidation of hydroquinone.

| Base/eq. | Isolated Yield [%] |
|---|---|
| 10 eq. KOH | 95 |
| 10 eq. NaOH | 38 |
| 6 eq. NaOH | 36 |
| 4 eq. NaOH | 11 |
| 2 eq. NaOH | 0 |

The results show that the yield obtained with 10 eq. NaOH were considerably lower as compared to 10 eq. KOH. While for NaOH, the yield increased with increasing amounts of NaOH, the yield obtained with 6 eq. NaOH is similar to that obtained with 10 eq. NaOH (36 and 38%, respectively), indicating a saturation effect even at such low yields as compared to KOH. Accordingly, the use of KOH instead of NaOH according to the present invention considerably increases the yield.

### Example 4: Influence of the Concentration of Hydrogen Peroxide

Next, the concentration of H₂O₂ was investigated. To this end, the batch process as described in Example 1 was modified regarding the concentration of hydrogen peroxide solution employed during the reaction protocol. Results are shown in Table 3 below:

TABLE 3
Influence of the concentration of H₂O₂ on the isolated
yield of the oxidation of hydroquinone to 2,5-dihydroxy-1,4-
benzoquinone di-potassium salt.

| wt % of employed | |
|---|---|
| H₂O₂ solution | Isolated Yield [%] |
| 50 | 95 |
| 40 | 76 |
| 30 | 72 |

These data show that all tested concentrations of H₂O₂ (at least 30 wt %) resulted in yields of more than 70%. However, highest yields were obtained with about 50 wt % solution of H₂O₂.

### Example 5: Re-Use of Mother Liquor with Addition of KOH

The mother liquor as obtained from the filtering step during the batch process (as described in Example 1) was analyzed by titration for its base content and was adjusted to a concentration of 50 wt % KOH by addition of solid KOH (purity 85 wt %). This solution was then reused for another DKBQ batch process employing a total amount of 10 equivalents of KOH, essentially as described in Example 1 (e.g., with addition of hydroquinone and H₂O₂ as described in Example 1). The yield of the obtained DKBQ was 90%, which is only a very slight decrease compared to the results obtained with pristine chemicals (c.f. Example 1, Table 1). Accordingly, the mother liquor may be supplemented with KOH and suitably re-used in reaction process of the invention.

### Example 6: Re-Use of Mother Liquor with Distillative Treatment

The mother liquor as obtained from the filtering step during the batch process (as described in Example 1) was analyzed by titration for its base content and was concentrated to a mass fraction of 50 wt % KOH by (distillative) removal of water under reduced pressure. This solution was then reused for another DKBQ batch process employing a total amount of 10 equivalents of KOH, essentially as described in Example 1 (e.g., with addition of hydroquinone and H₂O₂ as described in Example 1). The yield of the obtained DKBQ was 94-96%. A distillatively treated mother liquor therefore leads to the same DKBQ yield as compared to the pristine KOH solution (c.f. Example 1, Table 1). In other words, despite the use of recycled mother liquor, no decrease in the DKBQ yield was observed. Therefore, this treatment (removal of water) is surprisingly well suited to re-use the mother liquor for the reaction process of the invention.

### Example 7: Re-Use of Mother Liquor with Activated Carbon Filtration Treatment

To the mother liquor as obtained from the filtering step during the batch process (as described in Example 1), activated carbon was added and the mixture was stirred at distinct conditions as described in Table 4 below. The obtained suspension was then filtered. The resulting filtrate was analyzed by titration for its base content and was adjusted to a concentration of 50 wt % KOH by addition of solid KOH (purity 85 wt %). This solution was then reused for another DKBQ batch process employing a total amount of 10 equivalents of KOH, essentially as described in Example 1 (e.g., with addition of hydroquinone and H₂O₂ as described in Example 1). The specific parameters for this process and the yields of the obtained DKBQ are summarized in Table 4.

TABLE 4
Parameters for the treatment of the mother liquor with activated
carbon and resulting yields of DKBQ, when the thereby obtained
solution was used for a subsequent DKBQ batch process.

| Employed amount of | | | Isolated yield |
|---|---|---|---|
| activated carbon [wt % | | | of DKBQ from |
| relative to the mass of the | Temperature | Time | subsequent batch |
| employed mother liquor] | [° C.] | [h] | process [%] |
| 5 | 20 | 1 | 96 |
| 5 | 20 | 72 | 96 |
| 1 | 20 | 1 | 91 |
| 1 | 50 | 1 | 93 |

As can be concluded from Table 4, the treatment of the mother liquor with 5 wt % activated carbon results in the same yield as compared to the pristine KOH solution (c.f. Example 1, Table 1), independent of the carbon treatment time (1 or 72 h). Even lowering the amount to only 1 wt % activated carbon only slightly decreased the yield. Likewise, the treatment temperature did not significantly influence the yield, with a considerably higher temperature (50° C. instead of 20° C.) only slightly increasing the yield (from 91 to 93%). Therefore, the activated carbon treatment as well is surprisingly well suited for re-using the mother liquor for the reaction process of the invention.

1. A process for the manufacture of a compound according to formula (I) 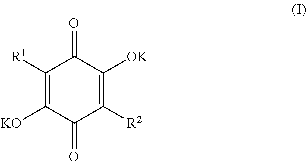
by alkaline oxidation of a compound according to formula (II) 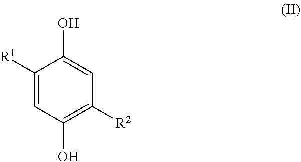
using KOH and H₂O₂,
wherein R¹ and R² are independently selected from the group consisting of —OR^{x}, —NO₂, —(NR^{x})R^{y}, —SR^{x}, —(SO)R^{x}, —(SO₂)R^{x}, —(SO₃)R^{x}, —(CO)R^{x}, —(CO₂)R^{x}, wherein R^{x} is H or C₁₋₆ alkyl and R^{y} is H or C₁₋₆ alkyl optionally substituted with —(CO₂)R^{x}; and salts thereof.

2. The process according to claim 1, wherein the process comprises the following steps:
(1) providing an aqueous solution of KOH;
(2) adding the compound of formula (II) to the aqueous KOH solution provided in step (1);
(3) adding H₂O₂ to the reaction mixture obtained in step (2); and
(4) incubating the reaction mixture at a temperature of at least 45° C.

3. The process according to claim 1 or 2, wherein the amount of KOH included in the reaction process is at least 8 equivalents relative to the amount of the compound of formula (II).

4. The process according to any one of the previous claims, wherein the amount of KOH included in the reaction process is at least 9 equivalents relative to the amount of the compound of formula (II).

5. The process according to any one of the previous claims, wherein the amount of KOH included in the reaction process is 9.8-12.2 equivalents relative to the amount of the compound of formula (II).

6. The process according to any one of the previous claims, wherein the amount of KOH included in the reaction process is about 10 equivalents relative to the amount of the compound of formula (II).

7. The process according to any one of the previous claims, wherein the H₂O₂ is provided in an aqueous solution of at least 27 wt % H₂O₂, preferably 30 wt % H₂O₂, more preferably at least 35 wt % H₂O₂.

8. The process according to any one of the previous claims, wherein the H₂O₂ is provided in an aqueous solution of at least 40 wt % H₂O₂.

9. The process according to any one of the previous claims, wherein the H₂O₂ is provided in an aqueous solution of at least 45 wt % H₂O₂.

10. The process according to any one of the previous claims, wherein R¹ and R² are independently selected from the group consisting of —H, —OH, and alkali-salts thereof.

11. The process according to any one of the previous claims, wherein each of R¹ and R² is —H.

12. The process according to any one of the previous claims, wherein the amount of H₂O₂ included in the reaction process is at least 2.5 equivalents relative to the amount of the compound of formula (II), preferably at least 2.7 equivalents, more preferably at least 3.0 equivalents.

13. The process according to any one of the previous claims, wherein the reaction temperature is at least 45° C., preferably at least 47° C., more preferably at least 50° C.

14. The process according to any one of the previous claims, wherein the concentration of KOH is 35-61 wt %, more preferably 37-57 wt %, even more preferably 40-55 wt %.

15. The process according to claim 14, wherein the concentration of KOH is 45±1 wt %.

16. The process according to claim 14, wherein the concentration of KOH is at least 50 wt %, such as 50-55 wt %.

17. The process according to any one of the previous claims, wherein essentially no NaOH is present during the oxidation.

18. The process according to any one of the previous claims, wherein essentially no organic solvent is present during the oxidation.

19. The process according to any one of the previous claims, wherein the sum of the weight of:
(i) the compound of formula (II);
(ii) KOH;
(iii) H₂O₂; and
(iv) water
is more than 95%, preferably more than 98%, more preferably more than 99% of the total weight of the reaction mixture at the beginning of the oxidation.

20. The process according to any one of the previous claims, wherein the process is a batch process.

21. The process according to any one of claims 1-19, wherein the process is a continuous process.

22. The process according to any one of the previous claims, wherein KOH is continuously added during the process.

23. The process according to any one of the previous claims, wherein H₂O₂ is provided to the reaction mixture via a venturi nozzle.

24. The process according to any one of the previous claims, wherein the reaction is cooled using a tube bundle heat exchanger.

25. The process according to any one of the previous claims, wherein the reaction is performed in a jacketed reactor.

26. The process according to any one of the previous claims comprising a step of isolation of the product from the reaction medium (mother liquor).

27. The process according to claim 26 comprising a further step of recycling or re-using the reaction medium (mother liquor) after isolation of the product.

28. The process according to claim 27, wherein the reaction medium (mother liquor) is directly used in a (further) process according to any one of the previous claims.

29. The process according to claim 27, wherein the reaction medium (mother liquor) is treated with activated carbon and, thereafter, used in a (further) process according to any one of the previous claims.

30. The process according to claim 27 or 29, wherein after isolation of the product the water in the reaction medium (mother liquor) is decreased and, thereafter, the resulting reaction medium is used in a (further) process according to any one of the previous claims.

31. The process according to any one of claims 27 to 30, wherein KOH is added to the reaction medium (mother liquor) before the reaction medium (mother liquor) is re-used in a (further) process according to any one of the previous claims.

32. The process according to any one of claims 27 to 31, wherein the reaction medium (mother liquor), optionally supplemented with KOH, is provided in step (1) of the process and steps (2)-(4) are performed as defined in any one of the previous claims.
